(19) United States
(12) Patent Application Publication
(10) Pub. No.: US 2014/0155821 A1
(43) Pub. Date: Jun. 5, 2014

(54) Infiltration Uterine Catheter Assembly

(71) Applicant: Cook Medical Technologies LLC, Bloomington, IN (US)

(72) Inventors: Jorge L. JIMENEZ-RIOS, Bloomington, IN (US); Matthew J. TERWISKE, Bloomington, IN (US)

(73) Assignee: Cook Medical Technologies LLC, Bloomington, IN (US)

(21) Appl. No.: 14/089,197

(22) Filed: Nov. 25, 2013

Related U.S. Application Data
(60) Provisional application No. 61/731,603, filed on Nov. 30, 2012.

(51) Int. Cl. A61M 25/01

(52) U.S. Cl. CPC A61M 25/0113
USPC 604/95.01; 604/528

(57) ABSTRACT

The present disclosure relates to a medical device. In one aspect, the medical device includes a catheter assembly. The catheter assembly includes a first catheter having a proximal end, a distal end, and a lumen disposed therethrough and a second catheter slidably disposed within the lumen of the first catheter, the second catheter having a distal end and a lumen disposed therethrough. The second catheter is moveable from a first position to a second position. A cervical seal is connected to the first catheter and the second catheter. A handle is engaged with the catheter assembly. In the first position, the cervical seal has a first outer diameter. In the second position, the cervical seal has a second outer diameter greater than the first outer diameter to create a seal in the cervix.

### RELATED APPLICATIONS

The present application claims priority to U.S. Provisional Patent Application Ser. No. 61/731,603 filed Nov. 30, 2012, the entirety of which is hereby incorporated by reference.

### TECHNICAL FIELD

This invention relates to a device for expediting the non-invasive infusion of fluid into the uterine cavity. More specifically, the device provides a single step procedure for infusing fluid through a single catheter assembly and sealing the uterus at the internal os of the uterus with a seal in order to perform therapeutic and/or diagnostic procedures.

### BACKGROUND

A number of conditions require the non-invasive entry into the uterine cavity, for both therapeutic and diagnostic purposes. Such access is provided through the cervix, transvaginally for diagnostic purposes. Contrast media and/or image enhancing media may be injected into the uterine cavity and radiography, sonohysterography may be carried out, to, e.g., establish the outline of the uterine cavity and/or patency of the Fallopian tubes, to diagnose or determine the development of polyps and/or submucous myomas. For example, medical professionals may use sonohysterography, a technique employed for imaging the uterine cavity, to diagnose uterine bleeding. Sonohysterography is performed by threading a catheter transcervically into the uterine cavity and delivering saline into the uterine cavity. The infusion of saline into the uterine cavity distends the cavity to provide contrast to the lining of the uterus. Diagnosis of endometrial and uterine pathology is performed by ultrasound.

Non-invasive access to the Fallopian tubes is also utilized for the purposes of artificial insemination as well as diagnostic reasons. Typically, this procedure involves injecting sperm into the uterine cavity. While this procedure can be carried out under hysteroscopic, radiographic or ultrasonograpic control, the common denominator of all of these procedures is access through the cervical canal and insertion of multiple catheters into the tubes.

A number of devices have been designed for the purposes discussed above, however, most involve the use of multiple devices and thus generally require multiple procedural steps as well as utilizing sealing devices, such as balloons, which may increase the discomfort to the patient as well as requiring longer time for carrying out the procedure. For example, the use of multiple devices to perform sonohysterogrpahy may increase cramping of the uterus. The use of multiple devices to complete the procedure may also increase patient recovery time, increase the risk of injury to the patient, and may involve greater expense. In addition, a number of devices are designed to seal the uterine cavity at the external os of the cervix by expanding the cervix, which may allow for displacement of the sealing device during the ultrasound procedure.

### BRIEF SUMMARY

The present disclosure relates to a medical device. In one aspect, a medical device includes a catheter assembly. The catheter assembly includes a first catheter having a proximal end, a distal end, and a lumen disposed therethrough and a second catheter slidably disposed within the lumen of the first catheter, the second catheter having a distal end and a lumen disposed therethrough. The second catheter is moveable from a first position to a second position. A cervical seal is connected to the first catheter and the second catheter. A handle is engaged with the catheter assembly. In the first position, the cervical seal has a first outer diameter. In the second position, the cervical seal has a second outer diameter greater than the first outer diameter. In some embodiments, the cervical seal has a bulb-like configuration. In other embodiments, the second catheter of the catheter assembly is coaxially positioned within the first catheter. In other embodiments, a proximal end of the seal is connected to the first catheter and a distal end of the seal is connected to the second catheter.

In another aspect, a medical device includes a catheter assembly. The catheter assembly includes a first catheter having a lumen disposed therethrough and a second catheter slidably disposed within the lumen of the first catheter, the second catheter having a distal end and a lumen disposed therethrough, the second catheter being moveable from a first position to a second position. A cervical seal is connected to the first catheter and the second catheter, the cervical seal having a frame disposed within an interior surface. An opening is disposed in the distal end of the second catheter distal of the cervical seal. A handle is engaged with the catheter assembly, the handle comprising a switch in communication with the second catheter and a port in fluid communication with the lumen of the second catheter and the opening in the distal end of the second catheter. The cervical seal has an expanded configuration and a compressed configuration. The outer diameter of the cervical seal in the compressed configuration is greater than the outer diameter of the cervical seal in the expanded configuration. In some embodiments, the outer diameter of the cervical seal in the expanded configuration ranges from about 3 mm to about 5 mm, and the outer diameter of the cervical seal in the compressed configuration ranges from about 9 mm to about 12 mm.

In yet another aspect, a medical device includes a coaxial catheter assembly. The catheter assembly includes a first catheter having a distal end and a lumen disposed therethrough and a second catheter slidably disposed within the lumen of the first catheter, the second catheter having a distal end and a lumen disposed therethrough. A cervical seal having a bulb-like configuration is connected to the first catheter and the second catheter, the cervical seal configured to occlude the internal os of a cervix. An opening is disposed in the distal end of the second catheter distal of the seal. A handle is engaged with the catheter assembly, the handle comprising a port in fluid communication with the lumen of the second catheter and the opening in the distal end of the second catheter. The cervical seal has an expanded configuration when the second catheter is in a first position, the expanded configuration having a first outer diameter. The cervical seal has a compressed configuration when the second catheter is in a second position, the compressed configuration having a second outer diameter greater than the first outer diameter. The handle includes a switch in communication with the second handle configured to move the second catheter within the lumen of the first catheter between the first position and the second position.

In another aspect, a method for sealing the uterine cavity includes providing a medical device, the medical device having a first catheter having a proximal end, a distal end, and a lumen disposed therethrough; a second catheter slidably disposed within the lumen of the first catheter, the second catheter having a distal end and a lumen disposed therethrough, the second catheter being moveable from a first position to a second position; a cervical seal connected to the first catheter and the second catheter, the cervical seal having an expanded configuration and a compressed configuration; and a handle engaged with the catheter assembly; inserting the medical device into the uterine cavity, the second catheter being in the first position; positioning a distal end of the medical device in close proximity to the internal os of the uterus; and occluding the internal os of the cervix by moving the second catheter from the first position to the second position such that a proximal end of the cervical seal is in contact with the internal os of the uterine cavity, where in the first position, the cervical seal is in the compressed position and where in the second position, the cervical seal is in the expanded position.

In a further aspect, a method for diagnosing uterine health includes inserting a medical device into the uterine cavity, the medical device comprising, a first catheter having a proximal end, a distal end, and a lumen disposed therethrough; a second catheter slidably disposed within the lumen of the first catheter, the second catheter having a distal end and a lumen disposed therethrough, the second catheter being moveable from a first position to a second position; a cervical seal connected to the first catheter and the second catheter, the cervical seal having an expanded configuration and a compressed configuration; and a handle engaged with the catheter assembly; occluding the internal as of the cervix by moving the second catheter from the first position to the second position; discharging a medium from a syringe into the device and injecting the medium into the uterus; diagnosing the uterus using an electronic diagnostic tool; and removing the medium and removing the device; where a proximal end of the cervical seal is connected to the first catheter and a distal end of the cervical seal is connected to the second catheter distal of the opening.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

FIG. 1 shows a plan view of an embodiment of the device.

FIG. 2 shows an embodiment of a handle for use with embodiment of the device of FIG. 1.

FIG. 3 shows a cross-sectional view of an embodiment of a cervical seal for the embodiment of the device of FIG. 1.

FIG. 4A shows a plan view of an embodiment of the device of FIG. 1 where an inner catheter diameter is in a first position.

FIG. 4B shows a plan view of an embodiment of the device of FIG. 1 where an inner catheter is in a second position.

FIG. 5 shows a schematic, front view of the intrauterine cavity and the device of FIG. 1.

FIG. 6 shows another schematic, front view of the intrauterine cavity and the device of FIG. 1.

FIG. 7 shows schematic, front view of the intrauterine cavity and the device of FIG. 1.

FIG. 8 shows an alternative embodiment of a cervical seal for use with an embodiment of the device.

FIG. 9 shows an alternative embodiment of a cervical seal for use with an embodiment of the device.

### DETAILED DESCRIPTION OF THE DRAWINGS AND THE PRESENTLY PREFERRED EMBODIMENTS

The embodiments of the medical device provide an effective, safe non-invasive procedure for instilling fluid into the uterine cavity for various diagnostic and/or therapeutic purposes. Embodiments of the device may be particularly useful for instilling saline into the uterine cavity and sealing the uterine cavity after the infusion of saline in order to enhance the ultrasound image. The present invention is not limited to those illustrated embodiments; it specifically contemplates other embodiments not illustrated but intended to be included in the claims.

Unless defined otherwise, all technical and scientific terms used herein have the same meaning as commonly understood by one of ordinary skill in the art to which this invention pertains.

The terms “patient,” “subject,” and “recipient” as used in this application may refer to any animal, particularly humans.

The term “biocompatible” refers to a material that is substantially non-toxic in the in vivo environment of its intended use, and that is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause a significantly adverse, long-lived or escalating biological reaction or response, and is distinguished from a mild, transient inflammation which typically accompanies surgery or implantation of foreign objects into a living organism.

The term “medical device” means any object that is itself or that includes a component that is intentionally inserted into the body of a patient as part of a medical treatment, and that comprises a structure adapted for introduction into a patient. The medical device can be a tool, such as, without limitation, a catheter, a wire guide, a forceps, or a scissors used to affect a surgical procedure at and/or deliver a second medical device to a treatment site in a patient. An alternative medical device of the present invention is one that is commonly intended to be a permanent implant, such as a stent.

In the following discussion, the terms “proximal” and “distal” will be used to describe the opposing axial ends of the assembly, as well as the axial ends of various components thereof. The term “proximal” is used in its conventional sense to refer to the end of the assembly (or component thereof) that is the closest to the operator during use of the assembly. The term” distal” is used in its conventional sense to refer to the end of the assembly (or component thereof) that is initially inserted into the patient, or that is closest to the patient during use.

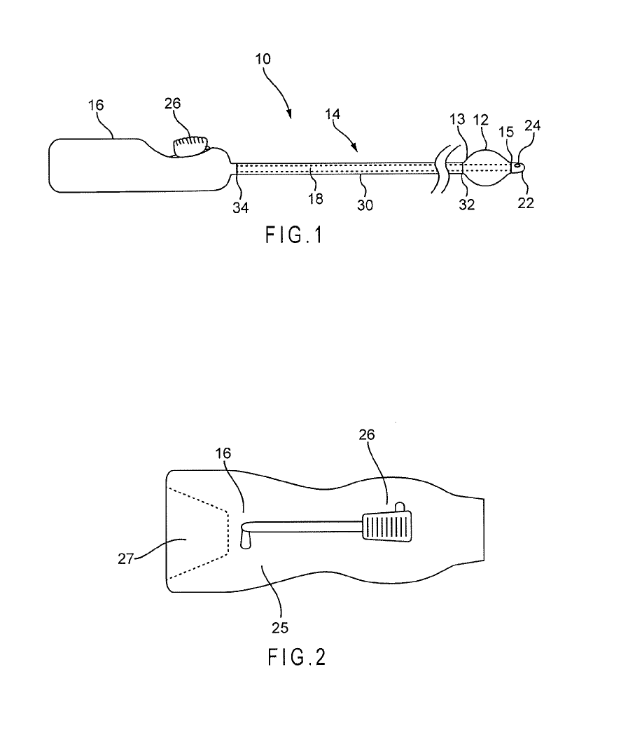

A more detailed description will now be given with references to FIGS. 1-9. FIG. 1 shows an aspect of an embodiment of a medical device 10. As shown, the medical device 10 includes a cervical seal 12, a catheter assembly 14, and a handle 16. The catheter assembly 14 comprises an inner catheter 18 and an outer catheter 30. The inner catheter 18 has a proximal end disposed within the handle 16 of the medical device 10 and a distal end 22. The inner catheter 18 has a generally elongate tubular configuration and has a lumen disposed within an interior surface of the inner catheter 18. The lumen of the inner catheter 18 is disposed throughout the entire length of the inner catheter 18. The proximal end of the inner catheter 18 may include a one-way valve to avoid producing air bubbles in the fluid to be infused into the uterus. The distal end 22 of the inner catheter 18 may be open or closed. As shown, the inner catheter has an opening 24 or port disposed just below the tip of the distal end 22. The opening 24 is in fluid communication with the interior lumen of the inner catheter 18 and a port 27 (as shown in FIG. 2) located on the handle 16. It can be appreciated by one of ordinary skill in the art that the lumen of the inner catheter 18 and the opening 24 on the distal end 22 of the inner catheter 18 may be used to introduce fluid, such as saline, into the body of the patient during a medical procedure. It will also be appreciated by one of ordinary skill in the art that the size of opening 24 may be configured based upon the needs of the physician.

The elongated tubular outer catheter 30 is at least partially disposed about an outer surface of the inner catheter 18. The outer catheter 30 includes a distal end 32 and a proximal end 34. As shown, the proximal end 34 of the outer catheter 30 is connected to the handle 16 of the medical device 10. A lumen is disposed within an interior surface of the outer catheter 30. The lumen of the outer catheter 30 may extend along its entire length. In one aspect, the inner catheter 18, as shown in FIG. 1, is at least partially coaxially positioned within the lumen of the outer catheter 30. The distal end of the outer catheter 30 may be connected to a proximal end 13 of the cervical seal 12.

The inner catheter 18 is sized to fit within the lumen of the outer catheter 30. In addition, the inner catheter 18 and the outer catheter 30 may be sized to fit within the particular part of the body for which the medical device 10 is being used. In one particular embodiment where the device is being placed within the uterus of a patient, the outer catheter 30 may have a size of 9 Fr and the inner catheter 18 may have a size of 5 Fr. It will be understood that the size of the inner catheter 18 and the outer catheter 30 may be determined based on the needs and dimensions of the patient. The inner catheter 18 and the outer catheter 30 are manufactured from biocompatible materials. In some embodiments, the inner catheter 18 and the outer catheter 30 are manufactured from the same materials. In other embodiments, the inner catheter 18 and the outer catheter 30 are manufactured from different materials. By way of example, and not by way of limitation, the inner catheter 18 and the outer catheter 30 may be an extrusion of any suitable materials such as (irradiated) polyethylene, nylon, tetrafluoroethylene, silicone, and polyurethane. In one embodiment, the outer catheter 30 is made from silicone and the inner catheter 18 is made from polyethylene. The inner catheter 18 is capable of telescopic movement from a first position and a second position within the lumen of the outer catheter 30. In particular, the operator may move the inner catheter 18 in either a proximal direction or a distal direction through the use of a switch 26 on the handle 16.

FIG. 2 shows an embodiment of a handle 16 for the medical device 10, the handle 16 comprises a main housing 25. A switch 26 is positioned on the main housing 25 and a proximal end (not shown) of the inner catheter 18 is in communication with the switch 26. The switch 26 is configured to control movement of the inner catheter 18 within the lumen of the outer catheter 30 in either a proximal direction or a distal direction. In present embodiment, the switch 26 may comprise a toggle-like switch. It will be understood by one of ordinary skill in the art that other types of switches may be used with the handle 16 of the medical device 10. The handle 16 may also include a locking mechanism to hold the switch 26 in the desired position during use of the medical device 10. A port 27 may be provided at the proximal end of the handle 16 in fluid communication with the proximal end of the inner catheter 18. The port 27 may be configured to receive other medical devices, such as a syringe, in order to introduce fluid into the medical device 10, and ultimately a patient, through the lumen of the inner catheter 18. In some embodiments, the port 27 may comprise a luer lock fitting to which a syringe may be secured. In other embodiments, a port 27 may be provided in a separate connector in fluid communication with the inner catheter 18 of the medical device 10.

Referring back to FIG. 1, the medical device 10 includes a cervical seal 12. The cervical seal 12 is positioned between the distal end 22 of the inner catheter 18 and the proximal end 34 of the outer catheter 30. A portion of the inner catheter 18 is coaxial positioned and disposed within an interior surface of the cervical seal 12. A distal end of the cervical seal 12 is attached to a distal portion of the inner catheter 18 proximate the opening 24 of the inner catheter 18. It will be appreciated by one of ordinary skill in the art that the distal end of the outer catheter 30 may be attached to the proximal end of the cervical seal 12 by numerous meetings including, but not limited to, the use of adhesives, mechanical methods such as soldering, etc. The cervical seal 12 is preferably produced from a biocompatible elastomeric material. Preferably, the cervical seal 12 is produced from a silicone rubber, e.g., SILASTIC ® Q7-4850 available from Dow Chemical. In some embodiments, the cervical seal 12, inner catheter 18, and the outer catheter 30 are separate components. In other embodiments, the cervical seal 12 and the outer catheter 30 may be integrally formed.

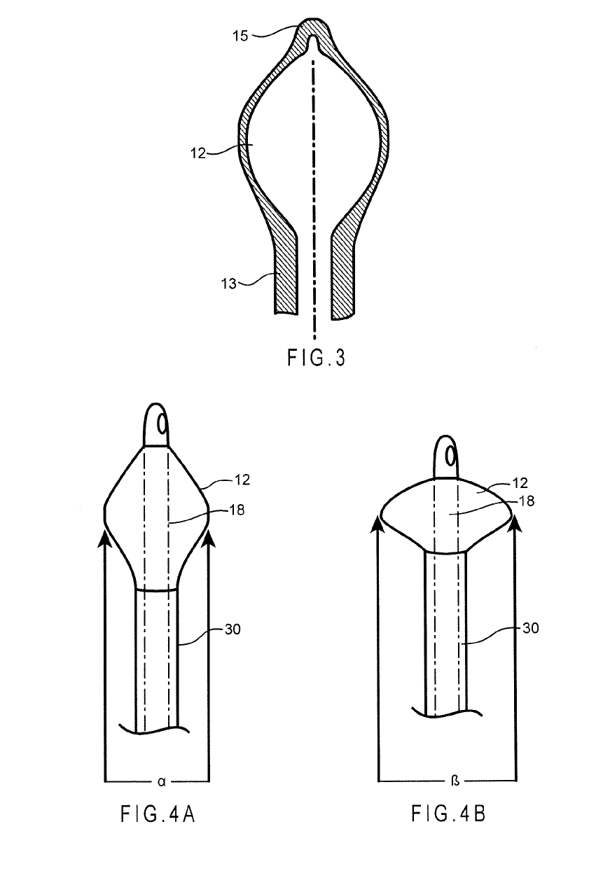

Referring to FIG. 3, an axial cross-section of the cervical seal 12 is presented. In this embodiment, the cervical seal 12 has a generally bulb-like configuration. The cervical seal 12 has a proximal end 13 and a distal end 15. As discussed above, the proximal end 13 of the cervical seal 12 may be attached to an outer surface of the outer catheter 30 and the distal end of the cervical seal 12 may be attached to an outer surface of the inner catheter 18. However, it will be recognized by one of ordinary skill in the art that the cervical seal 12 may have other configurations, including, but not limited to conical, spherical, cylindrical, etc. As shown, the cervical seal 12 may have a wall thickness a of about 0.5 mm.

FIGS. 4A and 4B show embodiments of the inner catheter in a first position and a second position, respectively. FIG. 4A shows the inner catheter 18 in the first position. To place the inner catheter 18 in the first position, the inner catheter 18 is moved distally through the lumen of the outer catheter 30 by moving the switch 26 on the handle 16 in a distal direction. The inner catheter 18 may be locked into the first position through the use of a locking mechanism. The cervical seal 12 is in an expanded configuration having a first outer diameter α in the radial direction. In particular, the length of the cervical seal 12 in the expanded configuration may range from about 20 mm to about 30 mm. In the expanded configuration, the first outer diameter α of the cervical seal 12 may range from about 3 mm to about 5 mm. With the inner catheter 18 in the first position, the medical device 10 may be inserted into the body of a patient and the cervical seal 12 will provide minimal interference upon placement into the body due to the reduced surface area of the cervical seal 12. For example, in a procedure requiring the occlusion of the uterus, the inner catheter 18 in the first position may be introduced by the physician into the uterus of the patient through the vaginal cavity. The cervical seal 12, in the expanded configuration, provides a smaller radial surface area and reduces the amount of interference the cervical seal 12 may cause to either the cervix or the uterine cavity when the medical device is in use.

Referring now to FIG. 4B, the inner catheter 18 is in the second position. To place the inner catheter 18 in the second position, the inner catheter 18 is moved proximally through the lumen of the outer catheter 30 by moving the switch 26 on the handle 16 in a proximal direction. The inner catheter 18 may be locked into the second position through the use of a locking mechanism. As shown in the figure, the cervical seal 12 is in a compressed configuration having a second outer diameter β in the radial direction, providing the cervical seal 12 with increased surface area. In the compressed configuration, the length of the cervical seal is decreased and providing the cervical seal with a second outer diameter β that is greater than the first outer diameter α of the cervical seal 12 in the expanded configuration. In particular, the length of the cervical seal 12 in the expanded configuration may range from about 10 mm to about 20 mm, preferably about 10 mm. The second outer diameter β may range from about 9 mm to about 12 mm. In a preferred embodiment, the cervical seal 12 may have a second outer diameter of about 10 mm and a length of 10 mm. With the inner catheter 18 in the second position, the medical device 10 may be used to occlude a region of the patient's body in order to allow for the introduction of fluid for performance of a medical procedure. For example, in a procedure requiring the occlusion of the uterus, the inner catheter 18 in the second position may be used to occlude the cervix of the patient at the internal os.

While the medical device 10 according to the invention may be utilized to deliver a therapeutic agent into the uterus, in its most preferable application, it is utilized for delivery of an image enhancement media, such as saline, for enhancing the image of an ultrasound image to detect abnormalities, polyps, tumors, determining and/or monitoring the thickness of the endometrial wall, and the like within the uterus. In addition, the cervical seal 12 provides an advantage by allowing for the accommodation of different and varying uterine dimensions. The ability to position the cervical seal 12 at the internal os allows for improved sealing of the cervix and helps to minimize any leaking of fluid from the uterus through the cervix during a procedure. In addition, the ability of the second catheter to move from a first position to a second position provides the physician with improved control of the cervical seal 12, which may allow the physician to adjust or remove the device more easily in the case of an emergency situation or patient discomfort.

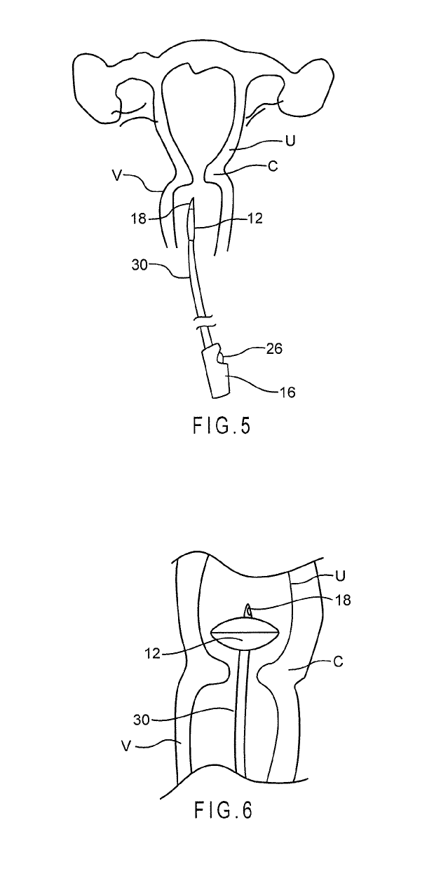

FIGS. 5-7 illustrate deployment of an embodiment of the medical device 10 into the uterus U of a patient. FIG. 5 depicts a schematic front view of the uterus U, cervix C, and vagina V. The medical device 10 is introduced into the body of the patient through the vagina V. As shown in the figures, the inner catheter 18 is in the first position and the cervical seal 12 is in the second position. While the inner catheter 18 and the cervical seal 12 are in these respective positions, the physician moves the device distally into the uterus of the patient through the cervical cavity. In order to prevent inadvertent movement of the inner catheter 18 from the first position to the second position, the physician may utilize the locking mechanism on the handle 16 to lock the switch 26 in position. Due to the reduced surface area and first outer diameter α of the cervical seal 12 in the expanded configuration, the cervical seal 12 does not interfere with the internal os, external os, or the walls of the cervix.

FIG. 6 depicts another schematic front view of the intrauterine cavity. The distal portion of the medical device 10 and the cervical seal 12 are shown in the uterus after transcervical placement. Once the distal end 22 of the inner catheter 18 and the cervical seal 12 are positioned within the uterus, the physician may move the inner catheter 18 from the first position to the second position by moving the switch 26 in a proximal direction. Movement of the inner catheter 18 into the second position moves the inner catheter 18 proximally within the lumen of the outer catheter 30. In addition, the proximal movement of the inner catheter 18 causes the cervical seal 12 to move from the expanded configuration to the compressed configuration. As shown in the Figures, the compressed configuration of the cervical seal 12 provides an increased maximum outer diameter for the cervical seal 12. The second outer diameter β of the cervical seal 12 provides an increased surface area to adequately seal the cervix at the internal os.

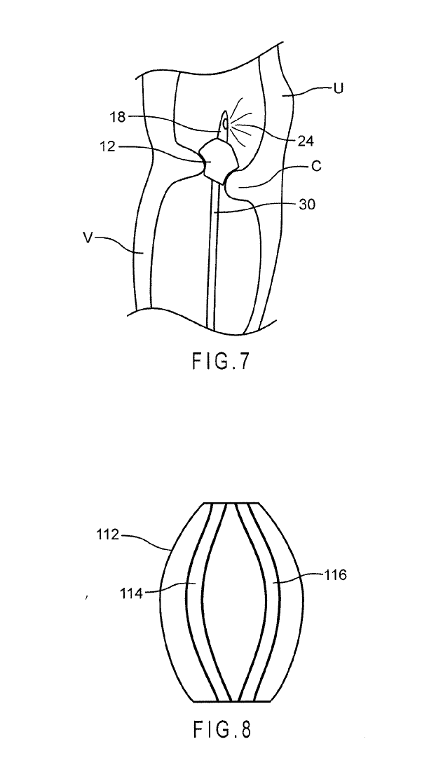

FIG. 7 depicts another schematic front view of the intrauterine cavity. The distal end of the inner catheter is shown positioned within the uterus. Once the cervical seal 12 has been placed in the compressed configuration, the physician may move the medical device 10 proximally such that a proximal portion of the cervical seal 12 is in contact with the internal os of the cervix. In this position, the cervical seal 12 occludes the cervix. Accordingly, a physician may introduce fluid into the uterus through the use of a device, such as a syringe, and the fluid will be prevented from exiting the uterus by the cervical seal 12.

To perform a sonohysterography, 5-10 ml (other amounts may be used) of saline, or other medically acceptable image contrast fluid is delivered from a syringe through the port 27 of the handle 16. The fluid travels through the lumen of the inner catheter 18 and exits the lumen through the opening 24. An ultrasound electronic diagnostic tool may be used to diagnose uterine health. Electronic diagnostic tools other than an ultrasound system may also be used to diagnose uterine health. These tools include, but are not limited to, an x-ray system, ultraviolet light system, or fluoroscopy system. An ultrasonic transducer may be used to generate images of endometrial and uterine pathology. In order to determine the cause of abnormal uterine bleeding or other uterine abnormality, the images of endometrial and uterine pathology are viewed on the ultrasonic image monitor, which is connected to the ultrasonic transducer. After an image is read using an ultrasonic transducer, saline is removed from the uterus by removing the cervical seal and allowing the saline to drain from the uterus.

FIG. 8 provides an alternative embodiment of the cervical seal 112 for use with a medical device. As shown, the cervical seal 112 has a generally bulb-like configuration. Furthermore, the cervical seal 112 includes a skeletal-like frame 114 disposed about an inner surface of the cervical seal 112. The frame 114 has a plurality of ribs 116 which extend about the entire length of the bulb. In other embodiments, the ribs of the frame 114 may only extend the midsection of the cervical seal 112 to the proximal end of the cervical seal 112. The ribs 116 of the frame have a Malecot-type configuration within the inner surface of the cervical seal 112. The frame 114 provides the cervical seal 112 with additional structural support, as well as, help to collapse the body of the cervical seal 112 evenly when it is placed in the compressed configuration. The frame 114 may be made from an extrusion of any suitable materials such as (irradiated) polyethylene, nylon, tetrafluoroethylene, silicone, and polyurethane. In addition, the frame 114 may be manufactured from biocompatible metals including, but not limited to, stainless steel and Nitinol. The frame 114 may be separately attached to the cervical seal 112 or may be made integral with the cervical seal 112.

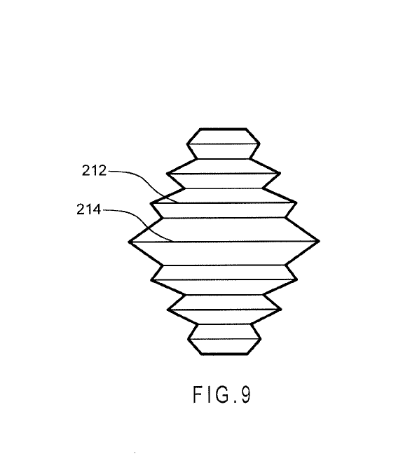

FIG. 9 provides an alternative embodiment of the cervical seal 212 for use with a medical device. As shown, the cervical seal 112 has a generally bulb-like configuration. Furthermore, the cervical seal 212 includes a series of folds 214 disposed throughout the length of the cervical seal 212. The folds extend axially about the surface of the cervical seal 212. The folds 214 include a variety of inward and outward folds in an alternating pattern. One example of an alternating pattern of inward and outward folds is often illustrated by an accordion. The folds 214 help to collapse the body of the cervical seal 212 evenly when it is placed in the compressed configuration.

A method for sealing the uterine cavity, the method comprising:

providing a medical device, the medical device comprising,
a first catheter having a proximal end, a distal end, and a lumen disposed therethrough;
a second catheter slidably disposed within the lumen of the first catheter, the second catheter having a distal end and a lumen disposed therethrough, the second catheter being moveable from a first position to a second position;
a cervical seal connected to the first catheter and the second catheter, the cervical seal having an expanded configuration and a compressed configuration; and
a handle engaged with the catheter assembly;

inserting the medical device into the uterine cavity, the second catheter being in the first position;

positioning a distal end of the medical device in close proximity to the internal os of the uterus;

and occluding the internal os of the cervix by moving the second catheter from the first position to the second position such that a proximal end of the cervical seal is in contact with the internal os of the uterine cavity;

where in the first position, the cervical seal is in the compressed position and where in the second position, the cervical seal is in the expanded position.

A method for diagnosing uterine health, the method comprising:

inserting a medical device into the uterine cavity, the medical device comprising,
a first catheter having a proximal end, a distal end, and a lumen disposed therethrough;
a second catheter slidably disposed within the lumen of the first catheter, the second catheter having a distal end and a lumen disposed therethrough, the second catheter being moveable from a first position to a second position;
a cervical seal connected to the first catheter and the second catheter, the cervical seal having an expanded configuration and a compressed configuration; and
a handle engaged with the catheter assembly;

occluding the internal os of the cervix by moving the second catheter from the first position to the second position;

discharging a medium from a syringe into the device and injecting the medium into the uterus;

diagnosing the uterus using an electronic diagnostic tool; and

removing the medium and removing the device;

where a proximal end of the cervical seal is connected to the first catheter and a distal end of the cervical seal is connected to the second catheter distal of the opening.

Throughout this specification various indications have been given as to preferred and alternative embodiments of the invention. However, the foregoing detailed description is to be regarded as illustrative rather than limiting and the invention is not limited to any one of the provided embodiments. It will be evident to one skilled in the art that modifications and variations may be made without departing from the spirit and scope of the invention. Changes in form and in the proportion of parts, as well as the substitution of equivalents, are contemplated as circumstances may suggest and render expedience; although specific terms have been employed, they are intended in a generic descriptive sense only and not for the purpose of limiting the scope of the invention set forth in the following claims. Moreover, the device is not limited to any specific dimension or material discussed above, nor is the device limited to being used with saline or an image contrast fluid alone.

1. A medical device, comprising:
a catheter assembly, the catheter assembly comprising:
a first catheter having a proximal end, a distal end, and a lumen disposed therethrough;
a second catheter slidably disposed within the lumen of the first catheter, the second catheter having a distal end and a lumen disposed therethrough, the second catheter being moveable from a first position to a second position;
a cervical seal connected to the first catheter and the second catheter;
a handle engaged with the catheter assembly;
where in the first position, the cervical seal has a first outer diameter and where in the second position, the cervical seal has a second outer diameter greater than the first outer diameter.

2. The medical device of claim 1, where the cervical seal has a bulb-like configuration.

3. The medical device of claim 1, where the first outer diameter of the cervical seal ranges from about 3 mm to about 5 mm.

4. The medical device of claim 1, where the second outer diameter of the cervical seal ranges from about 9 mm to about 12 mm.

5. The medical device of claim 1, where the cervical seal is integral with the first catheter.

6. The medical device of claim 1, where the cervical seal comprises a plurality of folds.

7. The medical device of claim 1, where the second catheter is coaxially positioned within the first catheter.

8. The medical device of claim 1, where a proximal end of the cervical seal is connected to the first catheter and a distal end of the cervical seal is connected to the second catheter.

9. The medical device of claim 1, where an opening is disposed in the distal end of the second catheter distal of the seal.

10. The medical device of claim 1, where the handle includes a switch configured to move the second catheter between the first position to the second position.

11. The medical device of claim 1, where the handle includes a port for introducing fluid.

12. A medical device, comprising:
a catheter assembly, the catheter assembly comprising:
a first catheter having a lumen disposed therethrough;
a second catheter slidably disposed within the lumen of the first catheter, the second catheter having a distal end and a lumen disposed therethrough, the second catheter being moveable from a first position to a second position;
a cervical seal connected to the first catheter and the second catheter, the cervical seal having a frame disposed within an interior surface;
an opening disposed in the distal end of the second catheter distal of the cervical seal, and
a handle engaged with the catheter assembly, the handle comprising a switch in communication with the second catheter and a port in fluid communication with the lumen of the second catheter and the opening in the distal end of the second catheter;
where the cervical seal has an expanded configuration and a compressed configuration and where the outer diameter of the cervical seal in the compressed configuration is greater than the outer diameter of the cervical seal in the expanded configuration.

13. The medical device of claim 12, where the cervical seal has a bulb-like configuration.

14. The medical device of claim 12, where the outer diameter of the cervical seal in the expanded configuration ranges from about 3 mm to about 5 mm.

15. The medical device of claim 12, where the outer diameter of the cervical seal in the compressed configuration ranges from about 9 mm to about 12 mm.

16. The medical device of claim 12, where the frame comprises a plurality of ribs extending longitudinally about the inner surface of the seal.

17. The medical device of claim 12, where the frame is integral with the bulb.

18. The medical device of claim 12, where a proximal end of the cervical seal is connected to the first catheter and a distal end of the cervical seal is connected to the second catheter distal of the opening.

19. The medical device of claim 12, where the cervical seal is in the expanded configuration when the second catheter is in the first position and where the cervical seal is in the compressed configuration when the second catheter is in the second position.

20. A medical device, comprising:
a coaxial catheter assembly, the catheter assembly comprising:
a first catheter having a distal end and a lumen disposed therethrough;
a second catheter slidably disposed within the lumen of the first catheter, the second catheter having a distal end and a lumen disposed therethrough;
a cervical seal having a bulb-like configuration connected to the first catheter and the second catheter, the cervical seal configured to occlude the internal os of a cervix;
an opening disposed in the distal end of the second catheter distal of the seal, and
a handle engaged with the catheter assembly, the handle comprising a switch in communication with the second catheter and a port in fluid communication with the lumen of the second catheter and the opening in the distal end of the second catheter;
where the cervical seal has an expanded configuration when the second catheter is in a first position, the expanded configuration having a first outer diameter,
where the cervical seal has an compressed configuration when the second catheter is in a second position, the compressed configuration having a second outer diameter greater than the first outer diameter, and
where the handle comprises a switch in communication with the second handle configured to move the second catheter within the lumen of the first catheter between the first position and the second position.
